(19) United States
(12) Patent Application Publication
(10) Pub. No.: US 2005/0163824 A1
(43) Pub. Date: Jul. 28, 2005

(54) Modified polymeric films

(76) Inventors: Victoria Jane Ayers, Cambridgeshire (GB); Jason Teckoe, Cambridgeshire (GB); Edward Zbygniew Nowak, Cambridge (GB)

Correspondence Address: MCDERMOTT WILL & EMERY LLP;ATTN: INTELLECTUAL PROPERTY DEPTARTMENT DOCKETING, 28 STATE STREET, BOSTON, MA, 02109, US

(21) Appl. No.: 10/514,514

(22) PCT Filed: May 12, 2003
(86) PCT No.: PCT/GB03/01996

(30) Foreign Application Priority Data
May 13, 2002 (GB) 0210859.5

(51) Int. Cl. A61K 9/70; A61K 9/48; A61K 31/716

(52) U.S. Cl. 424/443; 514/57; 424/451

(57) ABSTRACT

A hydroxypropyl methyl cellulose film comprises hydroxypropyl methyl cellulose plasticised with a plasticiser comprising an organic acid or a salt of an organic acid, preferably lactic acid, or an alcohol or salt of an alcohol. The film is safe for human consumption and finds use as a wall material of an ingestible delivery capsule, e.g. containing a dose of a pharmaceutical preparation.

### FIELD OF THE INVENTION

This invention relates to films of modified cellulose materials (or cellulose derivatives), particularly films of the modified cellulose material hydroxypropyl methyl cellulose (HPMC), and uses of such films.

### BACKGROUND TO THE INVENTION

Hydroxypropyl methyl cellulose is a synthetic plastics material, which is a modified form of the naturally occurring polymer cellulose. Films (or sheets or membranes) of HPMC are available commercially and have various uses, including proposals for use as wall materials of delivery capsules, i.e. capsules designed to retain and protect their contents until an intended site of delivery or conditions of delivery are encountered, at which point the contents of the capsules are released. HPMC is suitable for ingestion by humans, so delivery capsules with HPMC walls find potential use as ingestible capsules, e.g. for the delivery of accurately metered doses of pharmaceutical preparations and dietary supplements, as a possible replacement for gelatin-based capsules. See, for example, WO 97/35537, WO 00/27367 and WO 01/03676.

When producing HPMC films, HPMC is usually treated with a plasticiser in order to impart or improve properties of flexibility to the film. Materials used as plasticisers include polyethylene glycol (PEG), monopropylene glycol, glycerol and also acetins (which are acetates of glycerol).

In a typical method of making a cast HPMC film, HPMC, PEG and water are mixed to produce an aqueous solution, followed by optional deaeration of the solution if a non-aerated film is required. The solution is then fed in controlled manner to the surface of a continuous belt, producing a cast film of desired thickness which is fed on the belt past heating means for drying the film. The dried film is then removed from the belt and wound onto reels.

The present invention concerns novel plasticiser materials for HPMC films.

### SUMMARY OF THE INVENTION

In one aspect the present invention provides hydroxypropyl methyl cellulose film, comprising hydroxypropyl methyl cellulose plasticised with a plasticiser comprising a fruit acid or a salt of a fruit acid.

The term fruit acid is a term used in the cosmetics industry to refer to certain naturally occurring alpha hydroxy acids (AHAs) (i.e. acids with a hydroxyl group on the carbon atom immediately next to the carboxyl group), and this term is used in this specification to have this meaning. Fruit acids include the following:
1. Lactic acid (or 2-hydroxypropanoic acid), which is formed by fermentation of sugars and is found in milk.
2. Citric acid, which is found in the juice of citrus fruits, and in beets, cranberries and certain other acid fruits.
3. Malic acid (or hydroxysuccinic acid), which occurs in many acid fruits, e.g. grapes, apples and gooseberries.
4. Glycolic acid (or hydroxyethanoic acid), which occurs in the juice of sugar cane and beets.
5. Tartaric acid (or 2,3-dihydroxybutanedioic acid), which occurs in wine.
6. Hydroxy citric acid, which is found in the fruit of the Garcinia Gambogia tree.

Fruit acids, either naturally derived or synthetically produced, are all suitable for and approved for food use, so HPMC film in accordance with the invention is suitable for ingestion by humans. HPMC film in accordance with the invention can thus be used for ingestible purposes, e.g. as wall material for ingestible delivery capsules.

The currently preferred plasticiser is lactic acid, with citric acid then malic acid being the next most favoured.

It is preferred that the plasticiser is in the form of an acid rather than a salt of the acid as acids generally have better plasticising properties, although salts (including partial salts) e.g. sodium and potassium salts of the fruit acids may be used, and in particular it may be convenient to use buffered casting solutions.

The fruit acids, particularly lactic acid, are also generally found to have good plasticising properties and to be capable of producing HPMC films with certain benefits and advantages as compared with HPMC films prepared using conventional plasticisers. These benefits and advantages include the following:
a) The film thermoforms very easily, at lower temperatures and using less energy.
b) The deformed film retains its shape, i.e. the film has no memory.
c) The film readily welds to itself and seals at lower temperatures using less heat and pressure.
d) The film tastes pleasant and has a mouth watering effect.
e) The film has a high gloss appearance, improving the appearance of the finished produce.

The plasticiser may comprise one or more materials, including one or more fruit acids and/or one or more salts of a fruit acid, possibly in combination with one or more other plasticisers such as those used in the prior art, e.g. polyethylene glycol, monopropylene glycol, glycerol and acetins.

The plasticiser is suitably present in an amount in the range of 5 to 40% by weight of the total weight of the film, typically about 24% by weight of the total weight of the film. One preferred film thus comprises about 24% lactic acid and about 76% by weight HPMC. Where a mixture of plasticisers is used, benefits may nevertheless be seen using a fruit acid, particularly lactic acid, at lower levels, say 5% by weight of the total weight of the film.

The film may include optional colourings, e.g. in the form of known food dyes such as FD and C yellow number 5, optional flavourings, artificial sweeteners, textures etc., in known manner.

The film may optionally be foamed, expanded or gasified, with small pockets of gas, e.g. air, included in the film structure in known manner.

The film typically has a thickness in the range 50 to 200 microns, e.g. in the range 120 to 130 microns, with film thickness being controllable in known manner. Films of different thickness may be suited to different uses.

The film may be made in generally conventional manner, e.g. as described above, as is well known to those skilled in the art.

Film in accordance with the invention finds particular use as wall material for delivery capsules, as discussed above, particularly for ingestible capsules. Other uses include as biodegradable packaging, water-soluble sachets, carrier material for coating flavours (with flavour incorporated in the film or in coating on the film), for enrobing tablets etc.

In a further aspect the invention thus provides a delivery capsule having an enclosing wall comprising hydroxypropyl methyl cellulose film in accordance with the invention.

Such delivery capsules may be made in generally conventional manner, e.g. as disclosed in WO 97/35537, WO 00/27367 and WO 01/03676.

The invention will be further described, by way of illustration, in the following example.

### EXAMPLES

A hydroxypropyl methyl cellulose film in accordance with the invention was made, having the following composition by weight:

| | |
|---|---|
| Hydroxypropyl methyl cellulose | 76% |
| Lactic acid | 24% |

The film was made in generally conventional manner. HPMC, in the form of a powder, was mixed with lactic acid and water to produce an aqueous solution, with stirring. The composition of the HPMC casting solution was (% w:w) HPMC 10, water 86, lactic acid (80% solution) 4. The solution was deaerated by application of a vacuum. The solution was then fed to a feed hopper, including an elongate exit slot located a small distance above the upper surface of a moving conveyor belt adjacent an end thereof, with the slot extending perpendicularly with respect to the direction of movement of the belt. The feed arrangement geometry and speed of movement of the belt were such that a layer of liquid of desired thickness was applied to the belt and was moved on the belt away from the feed hopper, forming a film. The film was passed on the belt through a heating zone in which hot air heated the film, driving off water and so drying the film. The resulting dried, cast film was removed from the belt and wound onto reels. The water content of the dried film was about 4% by weight, in the form of bound (non-free) water. The thickness of the dried film was about 120 microns.

The film has certain benefits and advantages as compared with films prepared using conventional plasticisers. These include the following:
a) The film has a high gloss appearance on both sides, especially the air-dried side, improving the appearance of the finished product.
b) The film thermoforms very easily, at lower temperatures and using less energy.
c) The deformed film retains its shape, i.e. the film has no memory.
d) The film readily welds to itself and seals at lower temperatures (about 130° C. as compared with 160° C.) using less heat and pressure.
e) The film tastes pleasant and has a mouth watering effect.

The resulting film is suitable for human consumption, and one use is as a wall material for ingestible delivery capsules e.g. containing a dose of a pharmaceutical preparation or a dietary supplement. Such capsules may be made using known techniques, e.g. as described in WO 97/35537, WO 00/27367 and WO 01/03676.

1. A hydroxypropyl methyl cellulose film, comprising hydroxypropyl methyl cellulose plasticised with a plasticiser comprising an organic acid or a salt of an organic acid.

2. A film according to claim 1, wherein the plasticiser is a carboxylic acid.

3. A film according to claim 2 wherein, the plasticiser comprises one or more of maleic acid, fumaric acid, adipic acid.

4. A film according to claim 3 wherein the plasticiser comprises maleic acid.

5. A hydroxypropyl methyl cellulose film, comprising hydroxypropyl methyl cellulose plasticised with a plasticiser comprising an organic alcohol or a salt of an organic alcohol.

6. A film according to claim 5 wherein the plasticiser comprises the alcohol benzyl alcohol in combination with one or more of, maleic acid, fumaric acid, adipic acid.

7. A film according to claim 6 wherein the plasticiser comprises benzyl alcohol.

8. A film according to claim 1 wherein the plasticiser is present in the amount in the range 5 to 40% by weight of the total weight of the film.

9. A film according to claim 4 comprising about 23% by weight of the plasticiser and 77% by weight of HPMC.

10. A film according to claim 1, wherein the film is foamed, expanded or gasified.

11. A film according to claim 1 wherein the film has a thickness of between 50 to 200 microns.

12. A delivery capsule having an enclosing wall comprising a film in accordance with claim 1.

13. A method of producing HPMC film suitable for forming into a capsule, comprising treating the HPMC film with acids and/or alcohols mentioned in claim 1, before and/or during when the film is manipulated to form a capsule.

14. A film according to claim 5 wherein the plasticiser is present in the amount in the range 5 to 40% by weight of the total weight of the film.

15. A film according to claim 7 comprising about 23% by weight of the plasticiser and 77% by weight of HPMC.

16. A film according to claim 5, wherein the film is foamed, expanded or gasified.

17. A film according to claim 5, wherein the film has a thickness of between 50 to 200 microns.

18. A delivery capsule having an enclosing wall comprising a film in accordance with claim 5.

19. A method of producing HPMC film suitable for forming into a capsule, comprising treating the HPMC film with acids and/or alcohols mentioned in claim 5, before and/or during when the film is manipulated to form a capsule.
